(19) United States
(12) Patent Application Publication
(10) Pub. No.: US 2026/0183246 A1
(43) Pub. Date: Jul. 2, 2026

(54) Biodegradable Delivery Systems for Controlled Oral Delivery of Active Substances Using Microencapsulation and Multi-Trigger Mechanisms

(71) Applicants: Ashley Hurjak, Glendale, CA (US); Justin Polk, Playa Vista, CA (US); Daniel Gostanian, Las Vegas, NV (US)

(72) Inventors: Ashley Hurjak, Glendale, CA (US); Justin Polk, Playa Vista, CA (US); Daniel Gostanian, Las Vegas, NV (US)

(21) Appl. No.: 19/436,584

(22) Filed: Dec. 30, 2025

Related U.S. Application Data
(60) Provisional application No. 63/741,143, filed on Jan. 2, 2025.

(51) Int. Cl. A61K 9/50; A61K 9/00

(52) U.S. Cl. CPC A61K 9/5073; A61K 9/0056; A61K 9/501; A61K 9/5052; A61K 9/5068; A61K 9/5089

(57) ABSTRACT

An oral delivery system for at least one biologically active substance includes an orally insertable substrate device having surfaces. Microcapsules including the at least one biologically active substance adhere to the substrate device. The microcapsules define a core encapsulating the at least one biologically active substance. The microcapsules include multi-layer shells including a water-soluble adhesive layer and a pH-responsive layer and the microcapsules are adhered to the substrate surfaces for controlled release triggered by pH and fluid flow. In preferred embodiments the orally insertable device is a pacifier, straw or cutlery.

### CROSS REFERENCE TO RELATED APPLICATIONS

This application claims the benefit of priority to the following U.S. Provisional Patent Application: U.S. Provisional Application No. 63/741,143, filed Jan. 2, 2025, entitled Controlled-Release Electrolytes Adhered to Biodegradable Straws, the entirety of which is incorporated herein by reference.

### FIELD OF THE INVENTION

The present invention relates to biodegradable delivery systems for controlled release of active substances, including electrolytes, vitamins, minerals, medications, pharmaceuticals, nutraceuticals, proteins, essential amino acids, cannabinoids (such as CBD and THC), probiotics, and other bioactives. Specifically, the invention pertains to straws, utensils (e.g., spoons, forks, stirrers), and pacifiers fabricated from biodegradable materials and incorporating microencapsulated or nanoencapsulated payloads adhered via water-soluble, enzymatic, heat-activated, or dual-function adhesives. The systems utilize multi-layer encapsulation with pH-responsive, time-dependent, enzyme-triggered, temperature-sensitive, or friction-activated release mechanisms to enable targeted, sequential, or sustained delivery in oral, gastrointestinal, or other environments.

### BACKGROUND OF THE INVENTION

Conventional delivery mechanisms for active substances, such as pills, capsules, powders, and flavored beverages, suffer from limitations in convenience, precision, timing, and user compliance. Pills and capsules can be difficult to swallow, particularly for pediatric, geriatric, or special-needs populations, and often result in unpleasant tastes or inconsistent absorption. Alternative systems, like edible coatings on utensils or straws, frequently lead to premature release, degradation in neutral or mildly acidic liquids, or lack of targeted delivery.

Prior attempts to incorporate active ingredients into utensils or straws have resulted in issues such as premature dissolution, poor scalability, limited environmental sustainability, and insufficient versatility for diverse actives (e.g., hydrophobic cannabinoids or sensitive proteins). For instance, existing designs fail to provide robust adhesion mechanisms that prevent early release while ensuring controlled delivery in the stomach or other sites. Moreover, single-surface release systems do not allow for dual-phase or sequential delivery of multiple actives. Various relevant references include U.S. Pat. No. 11,517,872, titled “Encapsulation Method,” for its encapsulation processes suitable for microcapsule technology and U.S. Pat. No. 11,338,265, titled “Method for Preparing Microcapsules and Microparticles of Controlled Size,” for techniques to produce microcapsules with enhanced dissolution timing and precision.

Additionally, U.S. Publication No. 2005/0109858 A1 (Kerdar) discloses an administration form for oral delivery of active ingredients, vitamins, or nutrients using a drinking straw with a reversible bendable section acting as a closure device and a blocking device (e.g., stopper, membrane, or grid) to retain the formulation. The formulation is solid or highly viscous, often multi-particulate (50-1,500 μm particles), and is entrained by a conveying liquid during suction. However, this system lacks biodegradability in the straw material, does not employ microencapsulation or multi-layer shells for controlled release, and provides no mechanisms for dual-phase delivery (internal/external surfaces) or multi-trigger release (e.g., pH, enzyme, temperature, friction). It is limited to basic dissolution or suspension without advanced adhesion options or expansion to utensils and pacifiers, failing to address environmental sustainability, targeted delivery for sensitive actives, or versatility for hydrophobic compounds like cannabinoids.

U.S. Publication No. 2014/0084077 A1 (Knight) describes a drinking straw coated with water-soluble vitamins (B-12 and C) applied via an aqueous solution, resulting in a coating consisting solely of vitamins and water residue. The coating dissolves immediately upon contact with the beverage. This approach is deficient as it does not incorporate microencapsulation, biodegradability (though paper is mentioned, it is not emphasized), or controlled release mechanisms beyond simple dissolution. It lacks multi-layer structures, diverse adhesives, dual-phase delivery, and multi-triggers, and is restricted to specific vitamins without scalability to pharmaceuticals, proteins, or cannabinoids. Furthermore, it does not extend to utensils or pacifiers and provides no anti-workaround measures.

U.S. Publication No. 2018/0133108 A1 (Palazzi) teaches a drinking straw with an internal coating of a modified cellulose-based matrix (e.g., hydroxypropyl methylcellulose) dispersing active agents (e.g., sweeteners, flavors, nutrients, pharmaceuticals), with thickness <1 mm and dried to <5% water content. Release occurs progressively as liquid passes through. While offering some matrix-based release, this invention falls short in biodegradability of the straw (typically plastic), absence of multi-layer microencapsulation or nanoencapsulation, lack of external surface coatings for dual-phase delivery, and limited triggers (primarily dissolution-based, without enzyme, temperature, or friction activation). It does not include pacifiers, diverse adhesives (e.g., enzymatic), or applications for cannabinoids/proteins with enhanced stability via liposomes/hydrogels.

International Publication No. WO 2020/021111 A1 (Nolimal et al.) relates to a pre-filled straw for oral administration of pharmaceutical formulations, featuring granular particles (90% >500 μm and <2,000 μm) with a two-phase structure: a core (API, optionally coated) dispersed in a fast-dissolving matrix. The straw includes one-way valves for low-volume flushing (≤50 mL). Although it includes some coatings (e.g., enteric, taste-masking), it lacks comprehensive multi-layer encapsulation, biodegradability (materials not specified as biodegradable), dual-phase delivery, and expansion to utensils/pacifiers. The release described therein is primarily dissolution-based with limited triggers (e.g., no friction/temperature), and it does not incorporate nanoencapsulation, liposomes, or other measures for controlled release.

U.S. Pat. No. 5,718,681 (Manning) provides a medication delivery straw with a particle barrier at one end to retain dry or crushed medications, allowing fluid to dissolve and entrain them during suction. Optional features include funnels, caps, flexible necks, or medicine sacks. This system is inadequate as it omits biodegradability, microencapsulation, and controlled release mechanisms (relying on basic dissolution without multi-layers or triggers like pH/enzyme). It lacks dual-phase delivery, diverse adhesives, and extensions to pacifiers/utensils beyond basic straws, failing to ensure targeted release or stability for sensitive actives like proteins/cannabinoids.

U.S. Pat. No. 11,272,799 B2 (Palazzi) discloses a drinking straw with an internal coating of partially hydrolyzed guar gum (PHGG) matrix, optionally with acid and modified cellulose, dispersing active agents (e.g., vitamins, pharmaceuticals). The coating releases over 1-20 minutes in 100-500 mL liquid. While incorporating some biodegradable materials (e.g., PLA straws), it is limited by the absence of multi-layer encapsulation, external coatings for dual-phase, and multi-triggers (e.g., no enzyme/friction). It does not address pacifiers, or nanoencapsulation with liposomes for enhanced bioavailability or controlled release.

U.S. Pat. No. 11,925,603 B2 (Palazzi) describes a similar drinking straw with an internal hydroxypropyl methylcellulose matrix coating (<1 mm thick) for progressive release of active agents in 100-500 mL liquid. Deficiencies include no explicit biodegradability emphasis, lack of multi-layer microencapsulation, absence of dual-phase delivery or external surfaces, and restricted triggers (dissolution-only). It omits pacifiers/utensils, diverse adhesives, and advanced methods like electro spraying, and liposomal encapsulation of proteins and cannabinoids.

U.S. Publication No. 2005/0106187 A1 (Kerdar) teaches an administration form using a drinking straw with a cap featuring a pierceable or impact-removable bottom, a barrier device, and a multiparticulate formulation. It enables delivery via conveying liquid without spillage. However, it lacks biodegradability, microencapsulation/multi-layers, controlled triggers (beyond basic flow), dual-phase delivery, and extensions to pacifiers/utensils. It provides no nanoencapsulation limiting versatility for diverse actives.

### SUMMARY OF THE INVENTION

The present invention addresses these shortcomings by introducing biodegradable platforms with multi-layer microencapsulation, diverse adhesive mechanisms, and advanced encapsulation methods. These systems enable discreet, controlled dosing without altering beverage taste, while incorporating alternative triggers.

The inclusion of pacifiers expands applications to infant care, and the dual-surface (internal/external) design allows for simultaneous or staged release of actives like proteins and electrolytes.

The invention specifically provides biodegradable delivery systems comprising straws, utensils, and pacifiers with microencapsulated or nano encapsulated active substances adhered to internal and/or external surfaces via water-soluble, enzymatic, heat-activated, or dual-function adhesives. The microcapsules feature multi-layer structures, including: A first layer (e.g., water-soluble adhesive) for attachment; a second layer (e.g., pH-responsive coating) for targeted release in low-pH environments like the stomach; and optional additional layers (e.g., cellulose-based or hybrid) for delayed, sustained, or enzyme-triggered release.

Active substances include electrolytes (e.g., sodium, potassium, magnesium, calcium, chloride, bicarbonate, and phosphate, which enhance hydration by maintaining osmotic balance, supporting fluid retention, and facilitating nerve and muscle function), vitamins (e.g., water-soluble vitamins such as thiamine (B1), riboflavin (B2), niacin (B3), pantothenic acid (B5), pyridoxine (B6), biotin (B7), folate (B9), cobalamin (B12), and ascorbic acid (Vitamin C); these exhibit high bioavailability in aqueous solutions due to their solubility, with Vitamin C and B vitamins showing near-complete absorption when dissolved in water for rapid utilization in energy metabolism, immune support, and antioxidant activity), minerals, medications, pharmaceuticals, proteins (e.g., essential amino acids and signaling proteins such as vasopressin (antidiuretic hormone, ADH) and natriuretic peptides; in this context, vasopressin is most likely for hydration regulation as it signals the kidneys to reabsorb water, reducing urine output and maintaining body fluid levels, while natriuretic peptides promote sodium excretion to balance fluid volume), cannabinoids (e.g., delta-9-tetrahydrocannabinol (THC), tetrahydrocannabinolic acid (THCA), cannabidiol (CBD), cannabidiolic acid (CBDA), cannabinol (CBN), cannabigerol (CBG), cannabichromene (CBC), tetrahydrocannabivarin (THCV), and cannabigerolic acid (CBGA); acid forms like THCA, CBDA, and CBGA offer pain management through neuroprotective effects, anti-inflammation via cytokine modulation, and antioxidant properties by scavenging free radicals without psychoactive effects), probiotics (e.g., Lactobacillus acidophilus, Bifidobacterium bifidum, Lactobacillus rhamnosus, Bifidobacterium longum, and Saccharomyces boulardii, which enhance digestion by producing enzymes that break down fibers and improve gut barrier integrity, thereby releasing captive nutrients like vitamins and minerals for better absorption), flavor enhancers, and biologics (e.g., enzymes, peptides).

Encapsulation methods encompass nanoencapsulation, emulsification, spray-drying, layer-by-layer assembly, electro spraying, liposomes, and hydrogel-based techniques to enhance bioavailability, stability, and taste masking.

The systems support dual-phase delivery: internal surfaces for laminar flow-triggered release (e.g., electrolytes in beverages) and external surfaces for friction-or enzyme-activated release (e.g., proteins via oral contact). Biodegradable substrates are made from polyhydroxyalkanoates (PHA), polylactic acid (PLA), cellulose derivatives, or plant fiber composites, ensuring environmental sustainability.

Advantages include precise, targeted release to improve absorption and reduce side effects. Additional advantages include versatility for applications in sports hydration, medical dosing, nutritional supplements, pediatric care, and recreational wellness, scalability across utensil types and active combinations and an Eco-friendly design with no taste alteration or structural degradation (e.g., no “sogging” of straws).

It can be appreciated that the present invention can include a kit of parts having a fork, spoon, straw and/or pacifier all impregnated with electrolytes, flavor defect maskers (i.e. salt such as sodium chloride and others), and/or selected nutrients such as ascorbic acid and other trace nutrients described herein.

### BRIEF DESCRIPTION OF THE DRAWINGS

FIGS. 1-6 are various embodiments of the invention of an oral delivery system including a substrate device with at least one orally deliverable substance.

FIG. 1 is a perspective view of straw body with microcapsules adhered internally.

FIG. 2 is a perspective view of a straw body with microcapsules adhered externally.

FIG. 3 is a perspective view of a pediatric pacifier with a nipple and microcapsules on the nipple.

FIG. 4 is a perspective view of a fork with microcapsules on the teeth.

FIG. 5 is a perspective view of a spoon with microcapsules on the bowl.

FIG. 6 is a perspective view of a knife with microcapsules on the blade.

### DETAILED DESCRIPTION

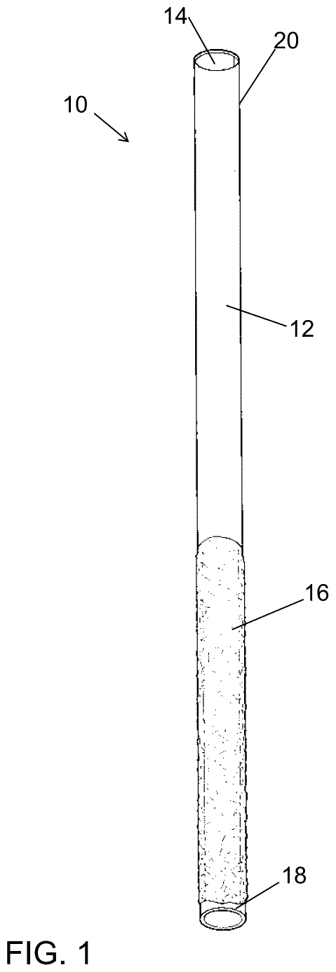

FIG. 1 is a substrate device including a drinking straw generally designated with the reference numeral 10. The straw 10 includes a surface 12 and an inner lumen 14. An orally deliverable substance 16 adheres to at least a portion of the straw 10. In particular, the substance 16 adheres circumferentially about the non-drinking end 18 of the straw 10. The drinking end 20 is free from the substance 16 in this embodiment.

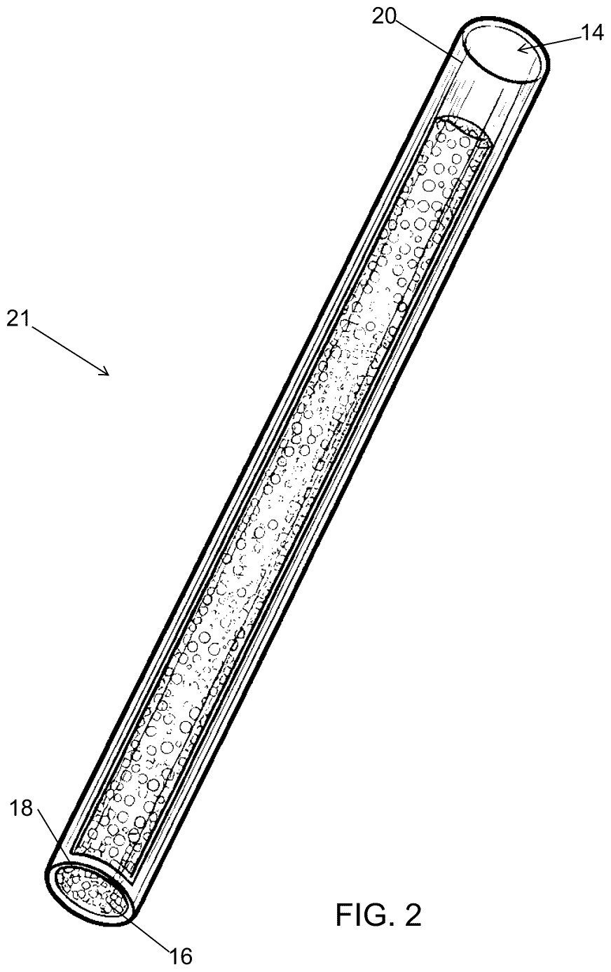

FIG. 2 is a drinking straw generally designated with the reference numeral 21. The straw 21 includes an orally deliverable substance 16 within the lumen 14 of the straw 10. In this embodiment, the substance 16 adheres about the inner circumference of the lumen 14 but does not fully fill the straw 21 to allow fluid flow between the drinking end 20 and non-drinking end18.

It can be appreciated that the straws 10 (FIG. 1) and the straw 21 can be integrated to have the deliverable substance 16 both adhering within the lumen 14 and on the outer circumference of the straw 21.

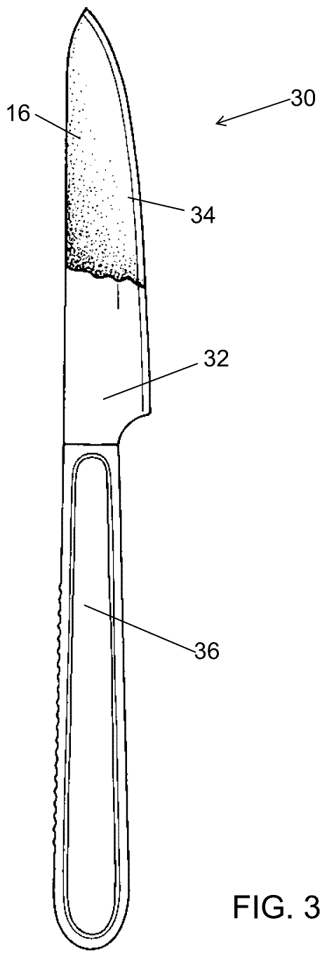

FIG. 3 is a knife generally designated with the reference numeral 30. The knife 30 is a common cutlery type knife, having a blade 32, a deliverable substance 16 adhered to the blade 32, and a handle 36 attached to the blade 32. Preferably, the knife 30 is disposable. The knife 30 is made from molded plastic and used for picnics, camping, restaurant service for fast food establishments, or other purposes. The deliverable substance 32 can be any of those described in this patent application, and may also include flavorings including table salt, pepper and other culinary spices.

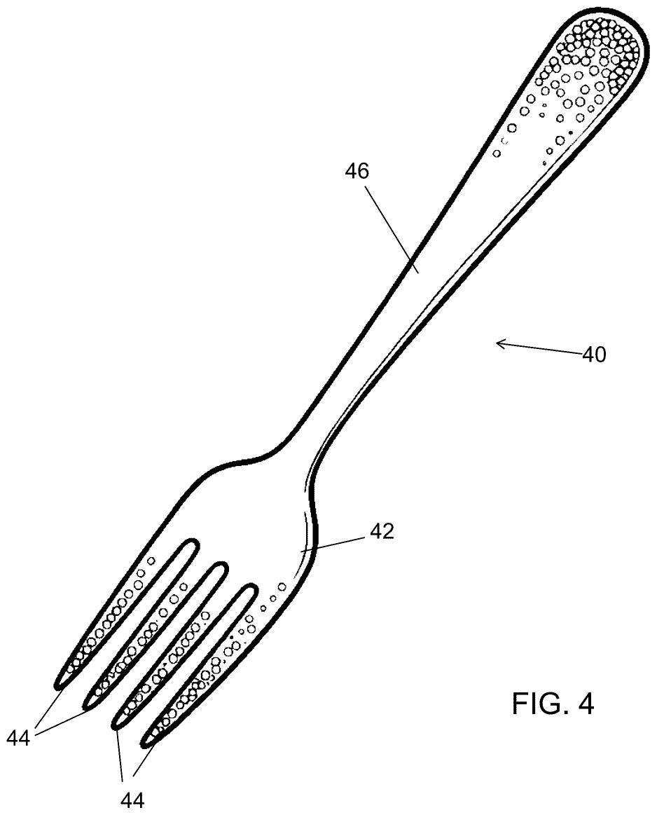

FIG. 4 is a fork generally designated with the reference numeral 40. The fork 40 has a forked end 42 with four teeth 44, and a handle 46 attached to the forked end 42.

A deliverable substance 16 adheres to both the forked end 42 on the teeth 44 and also on the handle 46. The deliverable substance 32 can be any of those described in this patent application, and may also include flavorings including table salt, pepper and other culinary spices.

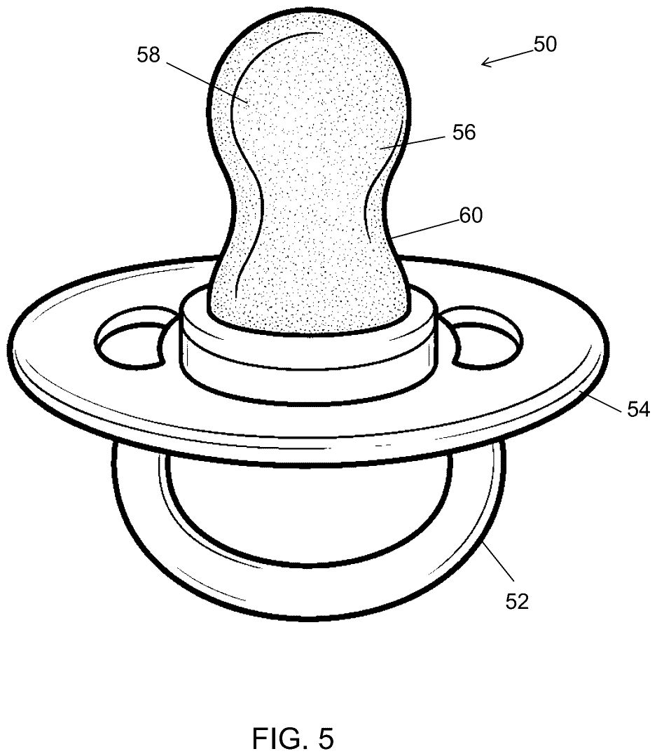

FIG. 5 is a pacifier generally designated with the reference numeral 50. The pacifier has a handle 52 attached to a plate 45 and a nipple 56. The plate 54 and handle are relatively rigid with respect to the nipple 56 which is softer and pliable. The deliverable substance 16 adheres to the nipple 56 on its outer periphery 60. This pacifier 50 is a pediatric device in this embodiment of the invention and the deliverable substance is preferably electrolytes that slowly releasee from the nipple 56 while a child has the nipple 56 inserted orally. The nipple 56 is deformable while sucked or chewed and the deliverable substance 16 adheres to the nipple 56. The deliverable substance 16 releases in response to mechanical motion, reaching a temperature threshold corresponding to the warmth of the oral cavity, and may also be responsive to saliva to effectuate release.

Other actives such as nutrients, flavorings and medicine can also be included in the deliverable substance 16, including those described in this document. Pain relief medications can be included to relieve teething discomfort.

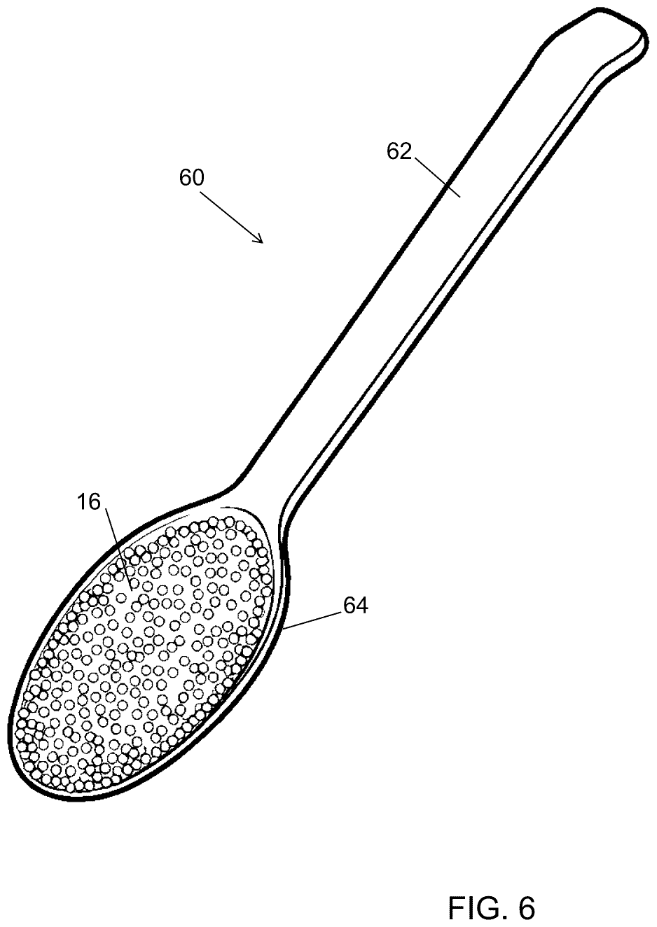

FIG. 6 is a disposable spoon generally designated with the reference numeral 60. The spoon 60 incudes a handle 62 and an operative end 64 having a concave cup. The deliverable substance 16 adheres to the operative end 64.

The devices of the oral delivery system of the present invention shown in FIGS. 1-6 preferably includes devices that are biodegradable and disposable. In addition to the drawings, the invention further includes stirrers, cups and cup lids engineered for controlled delivery of active substances.

The oral delivery system devices can be combined into a kit of parts including, for example, a straw 10, fork 40, spoon 60 and knife 30 wrapped in a napkin for food service use. A straw 10 or knife 30 can be considered a stirrer. A cup, in accordance with the present invention, is disposable and has deliverable substances adhered within the cup, or on the rim of the cup, or both.

These devices feature biodegradable substrate variations, including those constructed from compostable polymers such as Polyhydroxyalkanoate (PHA), Polylactic Acid (PLA), or plant fiber composites for strength and sustainability. These can be used for food service, home use, camping, picnics as well as institutional care, including medical care.

Straw designs include child-sized, large-diameter (e.g., for smoothies), or medical-grade variants. Straws can include a non-permeable boundary layer to inhibit chemical interaction between the straw and the active substances adhered thereto.

Utensils include spoons, forks, sporks and knives for functional dining, elderly care, or hospital nutrition.

Pacifiers with embedded or coated nipples for infant delivery, activated by sucking, saliva, or compression.

Microencapsulation Layers and Composition coat the utensils, pacifiers, and the straws externally, and in the case of straws, the coating can be either external, internal or both.

The microencapsulation layers contain active ingredients such as electrolytes like Na⁺, K⁺, Mg²⁺, Ca²⁺, Cl⁻, HCO₃⁻, and PO₄³⁻ to improve hydration. The layers can also include vitamins such as thiamine (B1), riboflavin (B2), niacin (B3), pantothenic acid (B5), pyridoxine (B6), biotin (B7), folate (B9), cobalamin (B12), and ascorbic acid (Vitamin C) and others with high aqueous bioavailability.

The layers can also include minerals; medications; proteins/essential amino acids including signaling proteins like vasopressin (ADH), which regulates hydration by promoting renal water reabsorption, and natriuretic peptides, which maintain fluid balance through natriuresis.

In one embodiment, the layers include select cannabinoids including tetrahydrocannabinol (THC), tetrahydrocannabinolic acid (THCA) (acid form for non-psychoactive pain relief and anti-inflammation), cannabidiol (CBD), cannabidiol acid (CBDA) (acid form with antioxidant and anti-inflammatory effects), cannabinol (CBN), cannabigerol (CBG) (anti-inflammatory), cannanbichromene (CBC), tetrahydrocannabivarin (THCV), and CBGA (acid form antioxidant).

In another embodiment, probiotics such as Lactobacillus acidophilus (enhances digestion of lactose and nutrient release), Bifidobacterium bifidum (improves gut barrier for better absorption), Lactobacillus rhamnoses (aids in breaking down fibers to free captive vitamins/minerals), Bifidobacterium longum, and Saccharomyces boulardii.

Biologics such as enzymes or peptides are actives in various embodiments of the invention. The peptides include both natural (e.g., collagen-derived peptides, casein hydrolysates) and synthetic forms (e.g., designed peptide sequences for specific functions), encapsulated for delivery and timed release.

Additionally, various other peptides that have the beneficial effect of hydration, such as analogues including sequences mimicking aquaporin channels to enhance cellular water uptake or peptides promoting mucin production for improved mucosal hydration, can be incorporated.

The peptides, in one embodiment, is encapsulated using alginate, its analogues (e.g., sodium alginate, calcium alginate), or related polysaccharides in natural or synthetic forms to achieve controlled, timed release while protecting against degradation.

Multi-Layer Shells are used in various embodiments of the invention including a first layer having a water-soluble adhesive (e.g., hydroxypropyl methylcellulose (HPMC), polyvinyl alcohol (PVA), mucilage, dextrin, alginate, honey-derived sugars, or gum arabic) for secure attachment to device external or internal surfaces.

A second layer adheres to the first layer and includes pH-responsive materials (e.g., Eudragit, alginate, chitosan) that dissolve in low-pH environments (e.g., stomach acid) for targeted release.

In various additional embodiments additional Layers adhered in sequence from the second layer include cellulose-based (e.g., methylcellulose, hydroxypropyl methylcellulose, carboxymethyl cellulose); hybrid (natural/synthetic polymers); or liposomes for protein, amino acid, or peptide stability and bioavailability. These layers can be designed to be pH-Responsive, Enzyme-Triggered (e.g., amylase/protease-activated), Temperature-triggered, Friction-Activated, or Time-Dependent layers for customized profiles and targeted delivery in various parts of the digestive tract.

The preferred adhesive mechanism of the first layer and other layers in straws are water-dissolvable to dissolve upon liquid contact, releasing microcapsules into beverages.

Enzyme-triggered actives can be activated by salivary/gastrointestinal enzymes (e.g., protein-based like casein or gelatin). Heat-Activated deliverables release in warm liquids, and within the body.

Dual-Function: Combines mechanisms (e.g., water +enzyme) for enhanced control.

Preferably, the active encapsulation method includes nanoencapsulation achieved via nanoprecipitation. Other encapsulation methods include ionic gelation, and lipid-based methods for hydrophobic actives (e.g., cannabinoids) to improve solubility and absorption.

Emulsification can include both single and double emulsions for hydrophilic and hydrophobic compounds.

Spray-drying atomizes solutions for uniform, nanosized capsules with taste masking (e.g., for electrolytes/salts). This method is particularly effective when combined with alginate for protein/peptide microencapsulation, protecting amino acids from oxidation and degradation while enabling delivery in beverages without altering flavor.

Layer-by-Layer Assembly can vary but generally includes alternating charged polymers for precise construction and delayed release.

Electrospraying uses electric fields for ultrafine capsules and the sizes can be engineered for desired delivery efficacy and timing.

Liposomes and hydrogels are used for sensitive proteins/biologics, protecting from oxidation/degradation and enabling buccal/GI absorption. Polymer microencapsulation uses poly(lactide-co-glycolide) (PLGA) or biopolymers for controlled/sustained release of pharmaceuticals and other actives including proteins and peptides.

Release mechanisms for straws include using the internal surface (lumen) and triggering it by laminar flow of aqueous fluid during use. Microcapsules detach from the lumen via adhesive dissolution and immediately dissolve into ingested liquid.

The external surface of various devices including straws are activated by oral friction, saliva, temperature, or enzyme activity for immediate or sequential release.

Dual-Phase Delivery is used for delivery of multiple actives simultaneously. For example, externally adhered probiotics and lumen delivered electrolytes can be delivered on a single straw simultaneously. Visibly colored THC can be delivered externally and CBD delivered via the lumen. This assumes nanoencapsulation of the cannabinoids to achieve water solubility.

Targeted release includes the use of microcapsules that remain intact until low pH (stomach acid) releases the actives from the microcapsules to ensure optimal absorption without premature degradation. Particular medicines, probiotics and peptides can be delivered to the small intestine directly after bypassing the gastric acids through slow degradation of the microcapsules.

Importantly, the present invention includes multi-trigger and combinable release mechanisms. The invention describes a range of alternative and combinable release triggers (e.g., pH-responsive, enzyme-triggered, temperature-sensitive, friction-activated, time-dependent, and laminar flow). This allows claims to cover not only individual triggers but also their combinations and functional equivalents.

It can be appreciated that the pH-triggered release can be alternated by substituting an enzyme trigger in various aspects of the invention. The broad disclosure supports claims encompassing such alternatives where substantially similar functions (controlled release in gastrointestinal environments) are achieved in substantially the same way (environmental stimuli dissolving layers) for the same result (targeted delivery). Both can be used in other aspects of the invention.

Adhesives are not limited to single types but include water-dissolvable, enzymatic, heat-activated, and dual-function variants (e.g., combining water solubility with enzymatic cleavage). This layered approach is applicable for various aspects of the invention. For instance, a dual-function adhesive (e.g., HPMC with casein) prevents simple substitutions, as altering one mechanism (e.g., avoiding water dissolution) still triggers the other (e.g., salivary enzymes).

The present invention's core technology accommodates a wide array of actives (e.g., electrolytes, proteins, cannabinoids, probiotics) and methods (e.g., nanoencapsulation, liposomes, layer-by-layer assembly). This invention prevents premature release unlike simple coatings, and is scalable to adapt for various deliverables, including particle sizes, various actives, and also medical and non-medical applications. The present invention supports simple ways for patients such as those with difficulty eating to safely consume cannabis, pharmaceuticals, electrolytes, and proteins. The present invention also has expanded applicability to pacifiers for pediatric dosing of electrolytes and other actives such as nutrients.

Preferably the present invention does not introduce flavor defects into the actives, and provides for discreet delivery of cannabinoids, and other medicines such as for consumers on airflights or in public arenas.

Electrolyte-loaded straws for athletes is one primary product for the present invention. Medication delivery for pill-averse patients and pacifiers for infants in need of vitamins/probiotics/meds is expected. Nutritional supplementation with vitamins, amino acids, or probiotics can be made easy and as routine as drinking and eating beverages and foods.

Cannabinoid-infused utensils are useful for discrete and controlled dosing.

Specialized care may benefit from peptide-infused spoons and the present invention can be used for delivering digestive aids such as lactase in the various inventive devices for enabling the digestion of lactase found in milk.

### ADDITIONAL EMBODIMENTS

Various embodiments with illustrative set forth in w/w ranges cover liposomal encapsulation, multi-layer systems, pH-responsive and enzymatic triggers, and straw/pacifier embodiments.

In one embodiment, an electrolyte formulation is encapsulated within a liposomal carrier and affixed to the internal lumen of a biodegradable straw. A representative composition on a w/w basis of the encapsulated composition includes an electrolyte blend comprising one or more sodium, potassium, and/or magnesium salts at about 60-85%, liposomal carrier components (phospholipids and optional sterols) at about 5-25%, and stabilizers or processing aids at about 0-10%. The liposomal formulation is applied to the straw lumen and retained using a water-soluble or saliva-soluble adhesive layer, with release occurring upon fluid flow during use.

In another embodiment, electrolytes are encapsulated within liposomes, which are further encapsulated within a polymeric shell to provide staged release. A representative composition on a w/w basis of the finished microcapsule includes a liposomal encapsulated electrolyte core at about 50-80%, a polymeric shell layer (alginate-based, chitosan-based, and/or pH-responsive polymer) at about 10-40%, and an optional outer adhesion or compatibilizer layer (e.g., a cellulose derivative) at about 0-15%. The liposomal core enhances stability and bioavailability, while the polymeric shell delays release until exposure to a trigger such as beverage flow or pH change.

In another embodiment, a liposomal formulation is applied to an external surface of a straw or utensil for release via saliva or friction during oral contact. A representative composition on a w/w basis of the surface coating includes one or more active ingredients (electrolytes, vitamins, proteins, or probiotics) at about 30-60%, a liposomal or hydrogel carrier matrix at about 20-50%, a saliva-responsive or enzyme-responsive polymer at about 5-20%, and an adhesive layer at about 0-10%. Upon oral contact, saliva or mechanical friction disrupts the carrier matrix, releasing the active ingredient in a controlled manner.

In another embodiment, a straw includes two distinct delivery zones. The internal lumen coating may include an electrolyte blend at about 60-85%, liposomal carrier components at about 5-25%, and stabilizers at about 0-10% (w/w of the lumen coating). The external surface coating may include a secondary active (e.g., a vitamin, protein, or probiotic) at about 20-60%, a liposomal, hydrogel, or microencapsulated carrier at about 20-50%, and an adhesive or protective layer at about 5-20% (w/w of the external coating). This configuration enables simultaneous or sequential delivery of multiple actives.

In another embodiment, the encapsulation systems described herein are integrated into a pacifier, bottle nipple, spoon, or similar oral-use device. A representative composition on a w/w basis of the encapsulated payload includes one or more active ingredients at about 10-50%, a liposomal and/or hydrogel carrier matrix at about 30-70%, and polymeric shell and adhesion layers at about 5-30%, with release occurring through repeated oral contact, fluid exposure, or mechanical agitation during use.

Preferably, the ratios may be adjusted depending on target dosing, release kinetics, and active stability. These ratios optimally vary by less than 20% for each component. Encapsulation methods may include liposomal encapsulation, nanoencapsulation, emulsification, spray-drying, or combinations thereof. Adhesion layers may be water-soluble, saliva-soluble, enzymatic, or pH-responsive. All examples are illustrative and non-limiting of the present invention. All ratios are expressed on a weight to weight w/w basis unless otherwise stated.

### SIMPLE EXAMPLES FOR RELEASE

Example 1 (Enzymatic Adhesive): Alginate-coated microcapsules with electrolytes affixed to a PHA straw via amylase-activated adhesive. Saliva triggers release for stomach absorption.

Example 2 (Nanoencapsulation): Double-emulsion PLGA capsules for THC; stable in water, dissolves in acid.

Example 3 (Multi-Layer Pacifier): Inner alginate (pH-sensitive)+outer cellulose (delayed); delivers vitamins during sucking. Flavors can also be added in layers.

Example 4 (Dual-Phase Utensil): Spoon with external protein coating (friction-release)+internal beta-alanine (flow-release) for sports recovery.

While the present invention is described in various embodiments, examples and broad suggestions, the appended claims precisely define the scope of the present invention.

We claim:

1. An oral delivery system for at least one biologically active substance comprising:
an orally insertable substrate device having surfaces;
microcapsules including the at least one biologically active substance adhered to the substrate device,
the microcapsules define a core encapsulating the at least one biologically active substance;
the microcapsules include multi-layer shells including a water-soluble adhesive layer and a pH-responsive layer, and
wherein the microcapsules are adhered to the substrate surfaces for controlled release triggered by pH and fluid flow.

2. The system of claim 1, wherein the adhesive is water-dissolvable, and selected from the group consisting of casein and gelatin.

3. The system of claim 1, wherein the adhesive is both heat-activated and dissolvable in an aqueous solution, the adhesive being selected from the group consisting of casein and gelatin.

4. The system of claim 1, further comprising an internal lumen and an exterior circumference, wherein the device has an internal lumen that releases the microcapsules in response to fluid flow and the external circumference releases the microcapsules in response to enzymes, to achieve a dual-phase release capability.

5. The system of claim 1, wherein the substrate devices is a pediatric pacifier with release activated by sucking or saliva.

6. A method of manufacturing the system of claim 1, comprising: forming the substrate; preparing microcapsules; adhering microcapsules using bioadhesives;
and applying layers for targeted release of the actives.

7. The system of claim 1, wherein the substrate device includes a nipple embedded with microencapsulated biologically active substance, and the nipple releases the biologically active substance via compression, pH, or enzyme triggers.

8. The system of claim 1, wherein the substrate device is a straw being infused with sodium, potassium and magnesium microcapsules for hydrating a user.

9. The system of claim 8, wherein the sodium, potassium and magnesium microcapsules are in a positive ionic form.

10. The system of claim 9, wherein the microcapsules encapsulate sodium chloride, potassium citrate, and magnesium glycinate.

11. The system of claim 9, wherein the system includes a beverage including glucose and the substrate device immerses in the beverage for consumption by a user.

12. The system of claim 8, wherein the straw includes an internal lumen and the at least one biologically active substrate adheres to within the lumen.

13. The system of claim 8, wherein the straw includes an exterior circumference and the at least one biologically active substance adheres to the circumference.

14. The system of claim 8, wherein the straw includes an internal lumen and an external circumference, and the at least one biologically active substance adheres to within the lumen and on the external circumference.

15. The system of claim 8, wherein the straw includes an internal lumen and an external circumference, and the at least one biologically active substance adheres to within the lumen and a second biologically active substance adheres to the external circumference to enable simultaneous delivery of the at least one biologically active substance and the second biologically active substance.

16. The system of claim 9, wherein the at least one biologically active substance is liposomally encapsulated and releases in response to fluid flow through the internal lumen and the second bioactive substance is liposomally encapsulated and released in response to immersion in an aqueous solution.

17. The system of claim 1, wherein the orally insertable substrate device is a drinking straw;
the drinking straw includes an external circumference and an internal lumen coated with the at least one biologically active substance, the at least one biologically active substance comprises:
an electrolyte blend in a concentration of about 60-85% w/w;
a liposomal carrier in a concentration of about 5-25% w/w; and
stabilizers in a concentration of about 0-10% w/w.

18. The system of claim 17, wherein the drinking straw external surface includes a second biologically active substance coating the external surface including:
includes an element selected from the group consisting of a vitamin, protein, probiotic, and combinations thereof in a total concentration of about 20-60% w/w of the second biologically active substance;
a carrier in a total concentration of about 20-50% w/w of the second biologically active substance, the carrier being selected from the group consisting of a liposome, hydrogel, and microencapsulated carrier, and combinations thereof;
an adhesive in a total concentration of at about 5-20% w/w of the second biologically active substance.

19. The system as set forth in claim 16, wherein the at least one biologically active substance includes electrolytes encapsulated within a first layer of liposomes, and further encapsulated within a second layer including a polymeric shell to provide staged release, the liposomal encapsulated electrolyte has a concentration of at about 50-80% w/w of the at least one biologically active substance, the polymeric shell layer is fabricated from elements in the group consisting of alginate, chitosan, and pH-responsive polymer, and combinations thereof;
the polymeric shell layer has a concentration of at about 10-40% w/w of the at least one biologically active substance, and
wherein the liposomal first layer enhances stability and bioavailability, and the polymeric second layer delays release of the electrolytes until exposure to a trigger such as beverage flow or pH change.

20. An oral delivery system for at least one biologically active substance comprising:
an orally insertable substrate device having surfaces including a handle, a plate and a nipple;
microcapsules including the at least one biologically active substance adhered to the nipple of the substrate device;
the microcapsules define a core encapsulating the at least one biologically active substance, and
the microcapsules include multi-layer shells including a water-soluble adhesive layer, the microcapsules release in response to compression.
